(19) United States
(12) Patent Application Publication
(10) Pub. No.: US 2023/0078141 A1
(43) Pub. Date: Mar. 16, 2023

(54) Abuse Alert Database (HAAD)

(71) Applicant: Joycelyn Cherise Adams, Long Beach, CA (US)

(72) Inventor: Joycelyn Cherise Adams, Long Beach, CA (US)

(21) Appl. No.: 17/476,979

(22) Filed: Sep. 16, 2021

(51) Int. Cl. G16H 40/20; A61B 5/00; G06Q 50/26

(52) U.S. Cl. CPC G16H 40/20; A61B 5/746; A61B 5/742; G06Q 50/265

(57) ABSTRACT

The Abuse Alert Database (HAAD), is a Specific Data Base for hospital. Urgent care, and nursing home use, allowing the admitting, ER staff, physicians and designated staff to be immediately alerted when victims of abuse or potential abuse present to various hospitals. The Database will be universal.

The Abuse Alert Database (for Hospitals, urgent care, and nursing homes) and handicap patients who may be victimized at the hands of care givers, will immediately alert admitting staff that patient(s) have been seen for moderate and specially for repeated visits to medical facilities for potential abuse or violence (Including minors with suffering from un ex-plained injuries, broken bones, bruises, head injuries, etc), at various other hospitals, and when necessary allow hospitals to use their discretion in contacting law enforcement regarding domestic and child abuse. In the case of minor it would expedite time as potential minor would be “flagged” as potential victims or abuse.

The app is designed because the majority majority of abuse victims when questioned by hospital staff or physicians leave and go to medical facilities or clinics they have not been to or have a history of going to that may alert staff to suspicious injuries or sicknesses brought on as a result of abuse. This will heighten hospitals awareness in a concerted effort to aid and hopefully save lives. This will save time by cutting staff having to look through antiquated or archived records to assist them in immediately knowing of abuse.

This Database will be for all medical facilities domestic and abroad (universal). The company developing the app will be granted the latitude needed to enhance the product as needed for the benefit of hospitals, clinics, and law enforcement.

Will be available to medical facilities and made to be easily installed for medical personnel.

### PURPOSE

### To Quickly Identify Victims of Abuse or Potential Abuse

The Abuse Alert Database (HAAD), is a Specific Data Base for hospital, urgent care and (nursing homes when patients present with unexplained injuries), allowing the Admitting, ER staff, physicians and designated staff to be immediately alerted when victims of abuse or potential abuse present to various hospitals. The Database will be universal.

The Abuse Alert Database (for Hospitals, Urgent Care, and nursing homes), and handicapped patients who may be victimized at the hands of care givers, will immediately alert admitting staff that patient(s) have been seen for moderate and specially for repeated visits to medical facilities for potential abuse or violence (Including minors suffering un ex-plained injuries, broken bones, bruises, head injuries, etc), at various other hospitals, and when necessary allow hospitals to use their discretion in contacting law enforcement regarding domestic and child abuse. In the case of minor it would expedite time as potential minor would be “flagged” as likely victim. (A flag potentially purple will appear on screen).

The app is designed because the majority of abuse victims when questioned by hospital staff or physicians leave and go to medical facilities or clinics they have not been to or have a history of going to that may be aware or suspicious of abuse. This will heighten hospitals awareness in a concerted effort to aid and hopefully save lives. This will save time by cutting staff having to look through antiquated or archived records to assist them in immediately knowing of abuse.

This Database will be for all medical facilities (universal). The company developing the app will be granted the latitude needed to enhance the product as needed for the benefit of hospitals, clinics, and law enforcement.

Will be available to medical facilities and made to be easily installed for medical personnel.

1. The Abuse Alert Database Hosp—(for Hospitals, Urgent Care, and nursing homes (facilities), and handicapped patients who may be victimized at the hands of care givers, will immediately alert admitting staff that patient(s) have been seen for moderate and especially for repeated visits to medical facilities for potential abuse and/or violence.
P (Including minors suffering un explained injuries, broken bones, bruises, head injuries, etc), at various other hospitals, and when necessary allow hospitals to use their discretion in contacting law enforcement regarding domestic and child abuse, In the case of minor it would expedite time as potential minor would be “flagged” as likely victim of abuse. (A tiny flag potentially purple will appear on screen) visible to medical personnel.
